(19) United States
(12) Patent Application Publication
(10) Pub. No.: US 2007/0010718 A1
(43) Pub. Date: Jan. 11, 2007

(54) Medical alert wear

(76) Inventors: James D. Spahn, Indianapolis, IN (US); James G. Spahn, Indianapolis, IN (US)

Correspondence Address: KRIEG DEVAULT LLP, ONE INDIANA SQUARE, SUITE 2800, INDIANAPOLIS, IN, 46204-2079, US

(21) Appl. No.: 11/410,529

(22) Filed: Apr. 25, 2006

Related U.S. Application Data
(60) Provisional application No. 60/674,575, filed on Apr. 25, 2005.

(51) Int. Cl. A61B 5/00; A61B 19/00

(52) U.S. Cl. 600/300; 128/898

(57) ABSTRACT

A method of providing medical alerts that are wearable, including the steps of providing a disposable wearable bracelet and printing on the bracelet an individual wearer's medical condition and the risk factors associated with the wearer's condition.

### BACKGROUND

The CMS (Center for Medicare/Medicaid Services) has designated the Continuum of Care as a high priority initiative. The Continuum of Care initiative enhances the necessity to create and transfer an individualized care plan in an uninterrupted fashion. This concept will be defined as the Seamless Continuum of Care Plan™.

The medical industry is evolving into a more rapid intrafacility and interfaculty transfer system. Because of this evolving system, the continuance of an individualized care plan has become increasingly more difficult to accomplish even though high technology means have been developed. While the rapidity of transfer from one treatment sign to another has grown, the availability of adequately trained professionals to prepare the patient for transfer and acceptance has diminished.

The problem of utilizing high tech systems is that locations both within and without the treatment facility may not be able to communicate medical information in a timely fashion. Certain disease entities, such as pressure ulcers, acute cardiac events, acute respiratory events and other life-threatening events, must be recognized and responded to in a timely fashion within minutes or hours for adequate medical outcomes to be accomplished.

An existing medical device called Medic Alert Bracelets are sold in the consumer market to individuals that possess certain ailments that need to be attended to in case of an emergency. The bracelet also has not only a 1-800 number for emergency medical records, but also has an emergency contact number, such as for a family member or guardian. Again, the services and information provided by the Medic Alert Bracelets are for emergency uses by an individual.

### SUMMARY OF THE INVENTION

The Medical Alert Wear of the present invention are distinctly different from Medic Alert Bracelets for a number of reasons. The first difference is that Medical Alert Wear is targeting medical facilities, not individuals. Medical Alert accessories are to be sold to facilities (long-term, short-term, homecare). This coincides with the Seamless continuum of Care Plan™. This process was developed to help maintain proper care and awareness of a patient throughout the many levels of care to insure recognition and proper care is given. Once a patient is admitted to a medical facility, the individual will be cared for by a number of doctors and caregivers. In many cases, certain problems are overlooked due to lack of communication by multiple floors, units, and facilities. Medical Alert accessories will stay with a patient throughout their stay to help the staff keep better track of an individual's specific medical problem. It is also important to note that patients are often transferred to other facilities, such as home care or nursing homes, and Medical Alert accessories will stay with the patient as long as the condition persists so as to allow a timely recognition and assessment of the medical condition that requires immediate attention.

The second distinguishing feature of Medical Alert Wear over Medic Alert Bracelets is in the information that is on the Medical Alert Wear accessories. Medical Alert Wear accessories have been created to not only list the medical problem but also to include the risk factors involved with that particular disease. For example, a pressure ulcer can have risk factors such as general medical condition, incontinence, immobility, existing pressure ulcer and prior ulcers. Each of these risk factors will be imprinted on the Medical Alert Wear accessory and will have the added feature of being marked to show which risk factor an individual possesses. The information provided on the Medical Alert Wear is far more beneficial than the Medic Alert Bracelet in a medical facility setting.

The final difference is that the Medical Alert Wear accessories are disposable. The Medic Alert Bracelet is not disposable and a new bracelet ($15) is required for each disease entity. The Medical Alert Wear accessories are disposable and inexpensive enough to through away in the event that a risk factor changes.

### BRIEF DESCRIPTION OF THE DRAWING

FIG. 1 is a perspective view of one preferred embodiment of the Medical Alert Wear by the present invention.

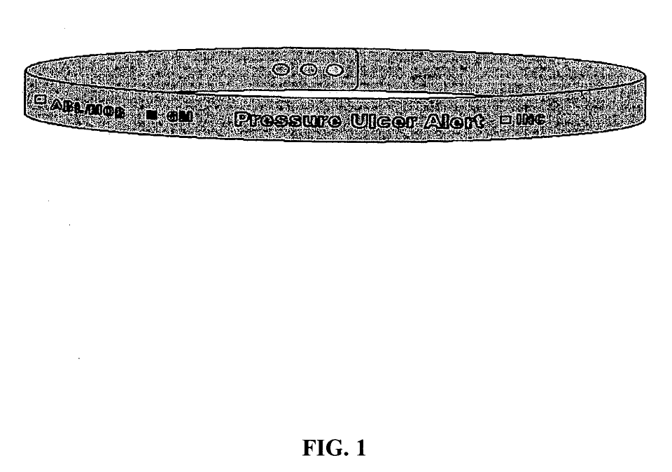

### DETAILED DESCRIPTION OF A PREFERRED EMBODIMENT

The Product

Recognizing the seriousness of the foregoing time sensitive services and the existing potential for inadequate information transfer is extremely important. The Medical Alert Wear accessories have been developed to help better recognize disease entities with timeliness and accuracy. This product line will encompass accessories such as, but not exclusively, bracelets, anklets, necklaces, buttons and broaches that can be made out of any non-latex material.

The accessories will be made out of any materials that are non-latex and approved by the FDA for hospital and medical use. The accessories will cover an array of medical entities that are not only problematic but may be fatal if the proper attention from the medical staff is delayed. Each accessory will focus on one specific medical entity and will have a list of major risk factors involved with that particular entity. These risk factors will help better define the scope of the medical entity. Next to each risk factor, an indented box will be present allowing the caregiver/clinician to mark specific (if not all) risk factors that pertain to the individual patient. The disease entity will be prominently displayed while the risk factors may be fully defined or abbreviated to match the size of the accessory. If abbreviated, an accompanying card will be available specific to each type of accessory explaining the abbreviations in detail.

Process

It is the intent to have the accessories placed on the patient by the first clinician who recognizes the disease entity. The clinician will assemble or mark the accessory so as to create awareness for future clinicians throughout the Seamless Continuum of Care Plan™. The choice of accessory will depend on cost, patient acceptability and ease of use. The accessory should then accompany the patient at all times to insure uncompromised awareness of the problem at subsequent levels of care. The disease entity of a pressure ulcer will be used as an example of how the Medical Alert Wear accessories will be utilized within the medical industry. The use of an adjustable bracelet or other accessory will have “Pressure Ulcer Alert” clearly labeled along with abbreviated risk factors that will be accompanied by an interpretation card for the abbreviations.

Once a clinician recognizes that a patient is at risk or currently has a pressure ulcer, the clinician will then place a Pressure Ulcer Medical Alert Alert Wear accessory on that patient. With a permanent marker, the clinician will fill in the appropriate indented check boxes that apply to the individual patient with a mark. This accessory will remain on the patient throughout the Seamless Continuum of Care Plan™. In the event that conditions should change, a clinician must mark any additional risk factors experienced by the patient. If an already marked condition is no longer valid, the clinician must remove the accessory and place a new, unmarked accessory on the patient and repeat the process.

The intent is that the Medical Alert Wear accessory be a visual, timely aware technique that in no way is intended to suggest or dictate policy and/or process that a facility has in place to address a particular disease entity.

It is important to note that a clinician may need written consent from a patient or a family member to apply the Medical Alert Wear accessory for compliance with the patient confidentiality act.

1. A method of providing medical alerts that are wearable, comprising the steps of
providing a disposable wearable bracelet;
printing on the bracelet an individual wearer's medical condition and the risk factors associated with the wearer's condition;
attaching the bracelet to the wearer.
